(19) United States
(12) Patent Application Publication
(10) Pub. No.: US 2025/0017859 A1
(43) Pub. Date: Jan. 16, 2025

(54) SOLID DISPERSION, PREPARATION METHOD, AND PHARMACEUTICAL COMPOSITION THEREOF

(71) Applicant: Anxo Pharmaceutical Co., Ltd., Taipei City (TW)

(72) Inventors: Chun-You LIOU, Taipei City (TW); Hsiang-Rong TSAI, Taipei City (TW); Tzu-Hsien CHAN, Taipei City (TW); I-Hsiang LIU, Taipei City (TW); Hua-Jing JHAN, Taipei City (TW); Tse-Hsien CHEN, Taipei City (TW); Chiung-Hui HUNG, Taipei City (TW); Chi-Heng JIAN, Taipei City (TW)

(21) Appl. No.: 18/768,908

(22) Filed: Jul. 10, 2024

Related U.S. Application Data
(60) Provisional application No. 63/525,960, filed on Jul. 10, 2023.

(51) Int. Cl. A61K 9/14; A61K 9/20; A61K 9/28; A61K 31/496

(52) U.S. Cl. CPC A61K 9/146; A61K 9/143; A61K 9/2009; A61K 9/2018; A61K 9/2054; A61K 9/28; A61K 31/496

(57) ABSTRACT

In some embodiments of the present disclosure, a solid dispersion is provided, comprising: lurasidone or its pharmaceutically acceptable salt and a carrier. The material of the carrier comprises polyvinyl acetate phthalate (PVAP), polyvinyl alcohol (PVA), cellulose acetate phthalate (CAP), mesoporous silica, hydroxypropyl methycellulose (HPMC), polyvinyl caprolactam-polyvinyl acetate-polyethylene glycol graft copolymer, acrylic resin, hydroxypropyl cellulose (HPC), povidone, copovidone, ethyl cellulose, polyoxyethylene glycol, hypromellose acetate succinate (HPMCAS), hypromellose phthalate (HPMCP), or a combination thereof.

### CROSS-REFERENCE TO RELATED APPLICATION

This application claims priority to U.S. Provisional Application Ser. No. 63/525,960, filed Jul. 10, 2023, which is herein incorporated by reference in its entirety.

### BACKGROUND

### Field of Invention

The present disclosure relates to solid dispersion, preparation method and pharmaceutical composition containing solid dispersion. In particular, the present disclosure relates to solid dispersion comprising lurasidone or its pharmaceutically acceptable salt.

### Description of Related Art

Lurasidone is atypical antipsychotics and approved for marketing in 2010 under the trade name Latuda®. The approved indication is schizophrenia and bipolar I disorder. As a biopharmaceutics classification system (BCS) class II medicine, lurasidone has the characteristic of high permeability and low water solubility, which is affected by pH. Compared with lurasidone in neutral medium, the solubility of lurasidone is much better in acidic medium.

Since Latuda® had obvious food effects, Latuda® should be administrated with food once daily. Specifically, compared with medicine administrated under fasting, the maximum plasma concentration (Cmax) and area under curve (AUC) of Latuda® administrated with food increased to 3 times and 2 times, respectively, which represents that bioavailability of medicine would be affected by eating (referred to as food effect).

For example, US patent U.S. Pat. No. 11,090,272 B2 discloses that Latuda® is an immediate release tablet. It is dissolved in stomach after administration. Since solubility of lurasidone changes along with pH, the large amount of lurasidone will precipitate in the intestine (neutral environment) and only a little part of lurasidone would be dissolved and absorbed. After the patient eats food, solubility of lurasidone improves by detergents secreted by gastrointestinal tract, so as to represent food effect.

However, even when Latuda® is administrated with food, the bioavailability of lurasidone is only 9-19%. In addition, there are also restrictions on calories intake that patients must eat more than 350 calories while administration, which leads to poor patient compliance.

Therefore, how to provide a dosage form of lurasidone that increases solubility of lurasidone in gastrointestinal tract (including neutral environment) for reducing food effect remains to be solved.

### SUMMARY

In one aspect of the present disclosure, a solid dispersion is provided, comprising: lurasidone or its pharmaceutically acceptable salt and a carrier. The material of the carrier comprises polyvinyl acetate phthalate (PVAP), polyvinyl alcohol (PVA), cellulose acetate phthalate (CAP), mesoporous silica, hydroxypropyl methycellulose (HPMC), polyvinyl caprolactam-polyvinyl acetate-polyethylene glycol graft copolymer, acrylic resin, hydroxypropyl cellulose (HPC), povidone, copovidone, ethyl cellulose, polyoxyethylene glycol, hypromellose acetate succinate (HPMCAS), hypromellose phthalate (HPMCP), or a combination thereof.

In some embodiments, a weight ratio of the lurasidone or its pharmaceutically acceptable salt to the carrier is from 3:1 to 1:30.

In some embodiments, a weight percentage of the lurasidone or its pharmaceutically acceptable salt is from 3% to 75%, and a weight percentage of the carrier is from 25% to 97% based on 100% by weight percentage of the solid dispersion.

In some embodiments, the carrier comprises a first carrier and a second carrier, the first carrier comprises the polyvinyl acetate phthalate (PVAP), the polyvinyl alcohol (PVA), the cellulose acetate phthalate (CAP), the mesoporous silica, the polyvinyl caprolactam-polyvinyl acetate-polyethylene glycol graft copolymer, the hydroxypropyl cellulose (HPC), the copovidone, the hypromellose acetate succinate (HPMCAS), or a combination thereof, and the second carrier comprises the polyvinyl alcohol (PVA), the mesoporous silica, the hydroxypropyl methycellulose (HPMC), the polyvinyl caprolactam-polyvinyl acetate-polyethylene glycol graft copolymer, the acrylic resin, the hydroxypropyl cellulose (HPC), the povidone, the copovidone, the ethyl cellulose, the polyoxyethylene glycol, the hypromellose acetate succinate (HPMCAS), the hypromellose phthalate (HPMCP), or a combination thereof.

In some embodiments, a weight ratio of the lurasidone or its pharmaceutically acceptable salt to the first carrier and the second carrier is from 3:1 to 1:30.

In some embodiments, a weight ratio of the first carrier to the second carrier is from 1:10 to 10:1.

In some embodiments, a weight percentage of the lurasidone or its pharmaceutically acceptable salt is from 3% to 75%, a weight percentage of the first carrier and the second carrier is from 25% to 97% based on 100% by weight percentage of the solid dispersion.

In some embodiments, the solid dispersion further comprises a surfactant, wherein the surfactant comprises glycerol esters, polyoxyethylene esters, or a combination thereof.

In one aspect of the present disclosure, a pharmaceutical composition is provided, comprising: the abovementioned solid dispersion and an excipient.

In one aspect of the present disclosure, a preparation method of a solid dispersion is provided, comprising: mixing lurasidone or its pharmaceutically acceptable salt and a carrier to form a mixture, in which the carrier comprises polyvinyl acetate phthalate (PVAP), polyvinyl alcohol (PVA), cellulose acetate phthalate (CAP), mesoporous silica, hydroxypropyl methycellulose (HPMC), polyvinyl caprolactam-polyvinyl acetate-polyethylene glycol graft copolymer, acrylic resin, hydroxypropyl cellulose (HPC), povidone, copovidone, ethyl cellulose, polyoxyethylene glycol, hypromellose acetate succinate (HPMCAS), hypromellose phthalate (HPMCP), or a combination thereof; and spray drying the mixture, or melting and then cooling the mixture.

In some embodiments, the step of mixing comprises mixing the lurasidone or its pharmaceutically acceptable salt and the carrier in an organic solvent to form the mixture.

In some embodiments, the step of melting comprises melting the mixture at a temperature of from 80° C. to 200° C. by using a hot melting extruder.

In one aspect of the present disclosure, a solid dispersion is provided, comprising: lurasidone or its pharmaceutically acceptable salt; a carrier; and a surfactant, in which the surfactant comprises glycerol esters, polyoxyethylene esters, or a combination thereof.

In some embodiments, a weight ratio of the lurasidone or its pharmaceutically acceptable salt to the carrier is from 3:1 to 1:30.

In some embodiments, a weight ratio of the lurasidone or its pharmaceutically acceptable salt to the surfactant is from 1:0.1 to 1:30.

In some embodiments, a weight percentage of the lurasidone or its pharmaceutically acceptable salt is from 3% to 75%, a weight ratio of the carrier is from 25% to 97%, and a weight ratio of the surfactant is from 0.1% to 45% based on 100% by weight percentage of the solid dispersion.

In some embodiments, the surfactant is fatty acid glycerides, polyoxyethylene fatty acid esters, or a combination thereof.

In some embodiments, the surfactant is polyoxylglycerides.

In some embodiments, a material of the carrier comprises polyvinyl acetate phthalate (PVAP), polyvinyl alcohol (PVA), cellulose acetate phthalate (CAP), mesoporous silica, hydroxypropyl methycellulose (HPMC), polyvinyl caprolactam-polyvinyl acetate-polyethylene glycol graft copolymer, acrylic resin, hydroxypropyl cellulose (HPC), povidone, copovidone, ethyl cellulose, polyoxyethylene glycol, hypromellose acetate succinate (HPMCAS), hypromellose phthalate (HPMCP), or a combination thereof.

In some embodiments, the carrier comprises a first carrier and a second carrier, the first carrier comprises the polyvinyl acetate phthalate (PVAP), the polyvinyl alcohol (PVA), the cellulose acetate phthalate (CAP), the mesoporous silica, the polyvinyl caprolactam-polyvinyl acetate-polyethylene glycol graft copolymer, the hydroxypropyl cellulose (HPC), the copovidone, the hypromellose acetate succinate (HPMCAS), or a combination thereof, and the second carrier comprises the polyvinyl alcohol (PVA), the mesoporous silica, the hydroxypropyl methycellulose (HPMC), the polyvinyl caprolactam-polyvinyl acetate-polyethylene glycol graft copolymer, the acrylic resin, the hydroxypropyl cellulose (HPC), the povidone, the copovidone, the ethyl cellulose, the polyoxyethylene glycol, the hypromellose acetate succinate (HPMCAS), the hypromellose phthalate (HPMCP), or a combination thereof.

In one aspect of the present disclosure, a preparation method of a solid dispersion is provided, comprising: mixing lurasidone or its pharmaceutically acceptable salt, a carrier and a surfactant to form a mixture, in which the surfactant comprises glycerol esters, polyoxyethylene esters, or a combination thereof; and spray drying the mixture, or melting and then cooling the mixture.

### BRIEF DESCRIPTION OF THE DRAWINGS

In order to make the above-mentioned and other objects, features, advantages and embodiments of the present disclosure more clearly understood, descriptions of accompanying drawings are as follows:

FIG. 1 illustrates a flow chart of a preparation method of a solid dispersion in some embodiments of the present disclosure.

FIG. 2 illustrates the release profiles of Experimental group 2-1 to Experimental group 2-3 and the commercial product, Latuda®, in the dissolution test in some embodiments of the present disclosure.

### DETAILED DESCRIPTION

In order that the present disclosure is described in detail and completeness, implementation aspects and specific embodiments of the present disclosure with illustrative description are presented, but those are not the only form for implementation or use of the specific embodiments of the present disclosure. The embodiments disclosed herein may be combined or substituted with each other in an advantageous manner, and other embodiments may be added to an embodiment without further description. In the following description, numerous specific details will be described in detail in order to enable the reader to fully understand the following embodiments. However, the embodiments of the present disclosure may be practiced without these specific details.

Although a series of operations or steps are described below to illustrate the method disclosed herein, the order of the operations or steps is not to be construed as limiting. For example, certain operations or steps may be performed in a different order and/or concurrently with other steps. In addition, not all illustrated operations, steps, and/or features are required to implement embodiments of the present disclosure. Moreover, each of the operations or steps described herein may include a plurality of sub-steps or actions.

### Definition

In this description, unless the context specifically dictates otherwise, “a” and “the” may mean a single or a plurality. It will be further understood that “comprise”, “include”, “have”, and similar terms as used herein indicate described features, regions, integers, steps, operations, elements and/or components, but not exclude other features, regions, integers, steps, operations, elements, components and/or groups.

As used herein, “active ingredient” refers to lurasidone or its pharmaceutical acceptable salts, including but not limited to salts, esters, complexes, chelating agents, cage compounds, racemates, mirror image isomers, or the like.

As used herein, “excipients” refers to pharmaceutical additives without pharmacological activity and used in pharmaceutical compositions according to different purposes and functions.

As used herein, “immediate release” (IR) refers to the phenomenon that the active ingredient is completely released in 2 hrs, 1 hr, 30 mins or less.

As used herein, “oral dosage form” refers to a way of oral administration, such as tablets.

### Solid Dispersion and Preparation Method Thereof

Lurasidone is insoluble in water (such as solubility in water is lower than 0.1 mg/mL) and has enhanced solubility in gastrointestinal tract while food uptake, which causes time point of drug administration is usually limited to food uptake, thereby decreasing patient compliance.

The main purpose of the present disclosure is to provide a solid dispersion including lurasidone or its pharmaceutically acceptable salt (active pharmaceutical ingredient, API) with improved solubility at least in neural condition, which reduces or eliminates food effects, thereby increasing patient compliance. Surprisingly, the solid dispersion of the present invention achieves higher solubility of lurasidone or its pharmaceutically acceptable salt by dispersing lurasidone or its pharmaceutically acceptable salt in the carriers to form solid dispersion and maintain lurasidone or its pharmaceutically acceptable salt in a non-crystalline form by selecting appropriate carriers suitable for pairing with lurasidone or its pharmaceutically acceptable salt. In addition, appropriate surfactant for increasing solubility of lurasidone or its pharmaceutically acceptable salt is also provided in the present disclosure.

By dispersing lurasidone or its pharmaceutically acceptable salt in the carrier, lurasidone or its pharmaceutically acceptable salt existed in an amorphous form to achieve the purpose of increasing solubility. The polymer chains of the carrier could be loosened through appropriate heating or dissolving in solvent during the preparation method, so that lurasidone or its pharmaceutically acceptable salt could be dispersed in the carrier. Therefore, the appropriate temperature and solvent are often considered at the same time, so that lurasidone or its pharmaceutically acceptable salt could be dispersed in the carrier in amorphous form.

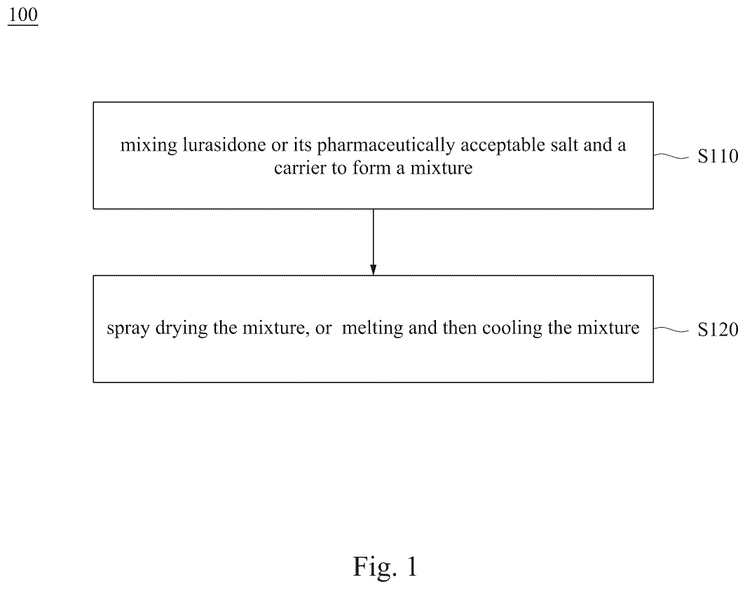

Please refer to FIG. 1, which represents a flow chart of a preparation method 100 of a solid dispersion in some embodiments of the present disclosure, comprising step S110 and step S120.

First of all, referring to step S110, lurasidone or its pharmaceutically acceptable salt and a carrier are mixed to form a mixture.

In some embodiments, step S110 comprises mixing the lurasidone or its pharmaceutically acceptable salt and the carrier in an organic solvent to form the mixture, in which the lurasidone or its pharmaceutically acceptable salt and the carrier can be evenly distributed in the organic solvent. In some embodiments, the organic solvent comprises dichloromethane, methanol, ethanol, dimethyl sulfoxide (DMSO), acetone, chloroform, isopropanol, or a combination thereof. In some other embodiments, step S110 comprises mixing the lurasidone or its pharmaceutically acceptable salt and the carrier in water.

In some embodiments, a material of the carrier comprises polyvinyl acetate phthalate (PVAP), polyvinyl alcohol (PVA), cellulose acetate phthalate (CAP), mesoporous silica, hydroxypropyl methycellulose (HPMC), polyvinyl caprolactam-polyvinyl acetate-polyethylene glycol graft copolymer, acrylic resin, hydroxypropyl cellulose (HPC), povidone, copovidone, ethyl cellulose, polyoxyethylene glycol, hypromellose acetate succinate (HPMCAS), hypromellose phthalate (HPMCP), or a combination thereof. It should be noted that the material of the carrier is selected after confirmed by practical tests (such as dissolution tests). Compared with the other common carrier material not selected in the present disclosure, the material of the carrier at least increases solubility of lurasidone or its pharmaceutically acceptable salt, and storage stability of solid dispersion, or both.

In some embodiments, the carrier comprises a first carrier and a second carrier, the first carrier comprises the polyvinyl acetate phthalate, the polyvinyl alcohol, the cellulose acetate phthalate, the mesoporous silica, the polyvinyl caprolactam-polyvinyl acetate-polyethylene glycol graft copolymer, the hydroxypropyl cellulose, the copovidone, the hypromellose acetate succinate, or a combination thereof, and the second carrier comprises the polyvinyl alcohol, the mesoporous silica, the hydroxypropyl methylcellulose, the polyvinyl caprolactam-polyvinyl acetate-polyethylene glycol graft copolymer, the acrylic resin, the hydroxypropyl cellulose, the povidone, the copovidone, the ethyl cellulose, the polyoxyethylene glycol, the hypromellose acetate succinate, the hypromellose phthalate, or a combination thereof. In some embodiments, the first carrier and the second carrier are different. That is, the carrier comprises at least two abovementioned materials. It is noted that compared with only one material, the solid dispersion of the present invention comprising at least two different carriers may achieve better solubility of lurasidone or its pharmaceutically acceptable salt in neutral medium or increased Cmax (maximum plasma concentration) and AUC (area under curve) in vivo study. In some embodiments, compared with other material, the carrier comprising PVAP or comprising PVA and mesoporous silica in the solid dispersion can achieve better solubility. In addition, the carrier comprising PVAP can further increase storage stability of lurasidone or its pharmaceutically acceptable salt.

In some embodiments, a weight ratio of the lurasidone or its pharmaceutically acceptable salt to the carrier (the first carrier and the second carrier) is from 3:1 to 1:30, such as 3:1, 2:1, 1:1, 1:5, 1:10, 1:15, 1:20, 1:25, 1:30 or any value between any interval of the abovementioned values. If the weight ratio of the lurasidone or its pharmaceutically acceptable salt is too low, large amount of the solid dispersion would be needed in the manufacturing of pharmaceutical composition comprising it. If the weight ratio of the lurasidone or its pharmaceutically acceptable salt is too high, the dispersed efficiency of lurasidone or its pharmaceutically acceptable salt is reduced, thereby decreasing the release efficiency of lurasidone or its pharmaceutically acceptable salt while administration.

In some embodiments, a weight ratio of the first carrier to the second carrier is from 1:10 to 10:1, such as 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1 or any value between any interval of the abovementioned values.

In some embodiments, step S110 comprises mixing the lurasidone or its pharmaceutically acceptable salt, the carrier and a surfactant to form the mixture, in which the surfactant comprises glycerol esters, polyoxyethylene esters, or a combination thereof. It should be noted that the surfactant is selected after confirmed by practical tests (such as dissolution tests) since the surfactant should be paired with lurasidone or its pharmaceutically acceptable salt and the foregoing procedure (the step of spray drying or the step of melting and cooling).

Through the addition of the selected surfactants, the miscibility of lurasidone or its pharmaceutically acceptable salt can be increased and the re-crystallization effect of lurasidone or its pharmaceutically acceptable salt can be reduced. Besides, the surfactant could improve the dissolution rate of lurasidone or its pharmaceutically acceptable salt and the wettability of the solid dispersion, thereby increasing bioavailability and avoiding precipitation of the solid dispersion.

In some embodiments, the surfactant comprises glycerol esters, polyoxyethylene esters, or a combination thereof. In some embodiments, the surfactant is fatty acid glycerides, polyoxyethylene fatty acid esters, or a combination thereof. Preferably, the surfactant is a combination of the fatty acid glycerides and the polyoxyethylene fatty acid esters. Preferably, the surfactant is polyoxylglycerides. In some embodiments, the polyoxylglycerides comprise caprylocaproyl polyoxylglycerides, lauroyl polyoxylglycerides, linoleoyl polyoxylglycerides, oleoyl polyoxylglycerides, stearoyl polyoxylglycerides, or a combination thereof.

In some embodiments, a weight ratio of the lurasidone or its pharmaceutically acceptable salt to the surfactant is from 1:0.1 to 1:30, such as, such as 1:0.1, 1:0.5, 1:1, 1:5, 1:10, 1:15, 1:20, 1:25, 1:30 or any value between any interval of the abovementioned values. If the weight ratio of the lurasidone or its pharmaceutically acceptable salt is too high, the dispersed efficiency of lurasidone or its pharmaceutically acceptable salt is reduced, thereby decreasing the release efficiency of lurasidone or its pharmaceutically acceptable salt while administration. If the weight ratio of the lurasidone or its pharmaceutically acceptable salt is too low, large amount of the solid dispersion would be needed in the manufacturing of pharmaceutical composition comprising the lurasidone or its pharmaceutically acceptable salt.

Furthermore, referring to step S120, the mixture is spray dried, or the mixture is melted and then cooled (such as Hot Melt Extrusion (HME)).

In some embodiments, the mixture is dissolved in spray drying solution (such as dichloromethane, methanol, ethanol, dimethyl sulfoxide (DMSO), acetone, chloroform, isopropanol, water or any combination thereof) before spray drying, in which lurasidone or its pharmaceutically acceptable salt is dispersed between the carriers in amorphous state. According to the step of spray drying, it should emphasized that since lurasidone or its pharmaceutically acceptable salt and each of the carrier and the surfactant have individual solubility in spray drying solution, the selected materials of the carrier and the surfactant should be paired with the solubility of the lurasidone or its pharmaceutically acceptable salt.

In some embodiments, after the mixture is dissolved in the spray drying solution, atomizing the spray drying solution comprising the mixture by airbrush in a drying room, in which the spray drying solution during atomizing will vaporize rapidly after contacting with hot dry gas, so as to obtain dry particles of the mixture. In some embodiments, an inlet temperature or an outlet temperature is from 35° C. to 200° C. (such as 35° C., 45° C., 50° C., 65° C., 70° C., 75° C., 80° C., 85° C., 90° C., 95° C., 100° C., 110° C., 120° C., 130° C., 140° C., 150° C., 160° C., 170° C., 180° C., 190° C., 200° C., or any value between any interval of the abovementioned values), in which the inlet temperature is higher than the outlet temperature. If the temperature is too high, the structure of lurasidone or its pharmaceutically acceptable salt, the carrier or the surfactant may be broken. If the temperature is too low, excessive residue of the spray drying solution remains.

According the step of melting and cooling, it should be emphasized that since lurasidone or its pharmaceutically acceptable salt and each of the carrier and the surfactant have individual working temperature scope while melting and cooling, the selected materials of the carrier and the surfactant should be paired with the working temperature of the lurasidone or its pharmaceutically acceptable salt.

In some embodiments, the step of melting and cooling comprises vacuum compression modeling (VCM) or hot-melt extrusion (HME).

Specifically, HME is a continuous process. During the process, the mixture are melted or soften through heat and pressure, and the molten mixture is evenly mixed. Then, the molten mixture is extruded through the holes of the outlet mold at the end of the machine and cooled to form extrudate. The heating temperature often above the glass transition temperature (Tg) of the ingredients of the mixture, sometimes above the melting point of lurasidone or its pharmaceutically acceptable salt so that all the ingredients can be mixed at the molecular level. Therefore, lurasidone or its pharmaceutically acceptable salt can be evenly dispersed in the carrier through the melting step. Furthermore, the extrudate can be cooled by air cooling, water cooling, etc to form solid dispersion.

In some embodiments, the step of melting comprises melting the mixture at a temperature of from 80° C. to 200° C. (such as 80° C., 90° C., 100° C., 200° C. or any value between any interval of the abovementioned values) by using a hot melting extruder. If the melting temperature is too high, the structure of lurasidone or its pharmaceutically acceptable salt, the carrier or the surfactant may be broken. If the melting temperature is too low, the distribution of lurasidone or its pharmaceutically acceptable salt is uneven. In some embodiments, the step of cooling comprises cooling the mixture at a temperature of from 20° C. to 30° C. (such as 20° C., 25° C., 30° C. or any value between any interval of the abovementioned values) to form solid dispersion.

After step 120, solid dispersion is then provided.

In some embodiments, a weight percentage of the lurasidone or its pharmaceutically acceptable salt is from 3% to 75%, such as 3%, 5%, 10%, 15%, 20%, 25%, 30%, 35%, 40%, 45%, 50%, 55%, 60%, 65%, 70%, 75% or any value between any interval of the abovementioned values, and a weight percentage of the carrier (the first carrier and the second carrier) is from 25% to 97% based on 100% by weight percentage of the solid dispersion, such as 25%, 30%, 35%, 40%, 45%, 50%, 55%, 60%, 65%, 70%, 75%, 80%, 85%, 90%, 95%, 97% or any value between any interval of the abovementioned values. If the weight percentage of the lurasidone or its pharmaceutically acceptable salt is too high or the weight percentage of the carrier is too low, the dispersed efficiency of lurasidone or its pharmaceutically acceptable salt is reduced, thereby decreasing the release efficiency of lurasidone or its pharmaceutically acceptable salt while administration. If the weight percentage of the lurasidone or its pharmaceutically acceptable salt is too low or the weight percentage of the carrier is too high, large amount of the solid dispersion would be needed in the manufacturing of pharmaceutical composition comprising the lurasidone or its pharmaceutically acceptable salt.

In some embodiments, when the solid dispersion comprises the surfactant, a weight ratio of the surfactant is from 0.1% to 45% based on 100% by weight percentage of the solid dispersion, such as 0.1%, 1%, 5%, 10%, 15%, 20%, 25%, 30%, 35%, 40%, 45% or any value between any interval of the abovementioned values. If the weight percentage of the surfactant is too low, the dispersed efficiency of lurasidone or its pharmaceutically acceptable salt is reduced, thereby decreasing the release efficiency of lurasidone or its pharmaceutically acceptable salt while administration. If the weight percentage of the surfactant is too high, large amount of the solid dispersion would be needed in the manufacturing of pharmaceutical composition since the weight percentage of lurasidone or its pharmaceutically acceptable salt may be too low.

In some embodiments, solubility of the solid dispersion in a medium of pH 6.0 including 0.01%-0.25% sodium dodecyl sulfate (SDS), such as 0.01% SDS, is more than 1 time of solubility of the lurasidone or its pharmaceutically salt, or even more than 2 times. The dissolution rate in a medium of pH 6.0 including 0.01%-0.25% SDS, such as 0.01% SDS, in 5-60 minutes is 1 time more than the control group of lurasidone tablet (Latuda®), or even more than 2 times.

In some embodiments, AUC of the lurasidone or its pharmaceutically salt in rats while the solid dispersion is administrated to rats under fasting is more than 1 time of lurasidone HCl, or even more than 1.5 times to 4 times. In some embodiments, Cmax of the lurasidone or its pharmaceutically salt in rats while the solid dispersion is administrated to rats under fasting is more than 1 time of lurasidone HCl, or even more than 1.5 times to 9 times. That is, the solid dispersion achieves better bioavailability than lurasidone HCl.

### Pharmaceutical Composition

A pharmaceutical composition comprising a solid dispersion is also provided in some embodiments of the present disclosure.

In some embodiments, the pharmaceutical composition is a solid dosage form. In some embodiments, the solid dosage form comprises powder, granule, fine granule, tablet or capsule.

In some embodiments, the pharmaceutical composition further comprises at least an excipient, such as filer, surfactant, disintegrant, lubricant or a combination thereof. In some embodiments, the filer comprises poly(methyl methacrylate), microcrystalline cellulose, methylcellulose, hydroxypropyl cellulose, hydroxypropyl methylcellulose, poly(ethylene oxide), polyoxypropylene, polyvinylpyrrolidone, carbomer, sodium carboxymethyl starch, carboxymethyl cellulose and sodium salt thereof, cross-linked sodium carboxymethyl cellulose, xanthan gum, lactose, starch, mannitol, pregelatinzed starch, corn starch, sorbitol, calcium sulfate, calcium hydrogen phosphate, calcium carbonate or a combination thereof. In some embodiments, the surfactant comprises sodium lauryl sulfate, quaternary ammonium compound, lecithin, fatty acid glycerides, polyoxyethylene esters, polyoxylglycerides, sorbitan fatty acid ester, polysorbate or a combination thereof. In some embodiments, the disintegrant comprises croscarmellose sodium, crospovidone, low-substituted hydroxypropyl cellulose, sodium starch glycolate, starch, pregelatinized starch or any combination thereof. In some embodiments, the lubricant comprises magnesium stearate, colloidal silicon dioxide, stearic acid, talc, glyceryl behenate, hydrogenated castor oil, sodium stearyl fumarate or a combination thereof.

In some embodiments, the dissolution rate of the pharmaceutical composition in a medium of pH 6.0 including 0.01%-0.25% SDS, such as 0.01% SDS, in 10-75 minutes is 1 time more than the control group of lurasidone tablet (Latuda®), or even more than 2 times to 4 times.

### Method for Treatment

In another aspect of the disclosure, a method of treating a mental disease is provided, comprising administering the abovementioned solid dispersion or the abovementioned pharmaceutical composition to a subject suffered from the mental disease. In some embodiment, the mental disease comprises schizophrenia, bipolar disorder, autism, depression or a combination thereof.

In another aspect of the disclosure, use of the abovementioned solid dispersion or abovementioned pharmaceutical composition in the manufacture of a medicament of treating a mental disease is provided. In some embodiment, the mental disease comprises schizophrenia, bipolar disorder, autism, depression or a combination thereof.

It should be understood that the above-described embodiments and the following examples are given by way of illustration, not limitation. Various changes and modifications within the scope of the present invention will become apparent to those skilled in the art from the present description.

### Example 1—Solid Dispersion

### Example 1.1—Preparation Method (Spray Drying)

The solvent evaporation preparation method of solid dispersion including lurasidone by spray drying is as follows:

Step 1, a spray drying solution is prepared according to the proportion listed in table 1.

Step 2, a pre-mixture of the active ingredient (lurasidone or lurasidone HCl) and the carrier (and the surfactant, optionally) are batch weighed according to the prescriptions in table 2 to table 6.

Step 3, the weighed pre-mixture in step 2 is added to the spray drying solution in step 1 to form a mixture.

Step 4, the mixture in step 3 is stirred until clear and then spray dried by using a spray dryer (Mini Spray Dryer B-290 (Büchi)). The spray drying parameters are shown in table 7.

Step 5, the granules obtained in step 4 (spray drying) are sieved through 60 mesh sieve to form solid dispersions of experimental groups 1 to 21, respectively.

TABLE 1
Spray drying solution

| | Experimental group | | | | | | | |
|---|---|---|---|---|---|---|---|---|
| | Group | 1-13 | 14 | 15 | 16 | 17 | 18 | 19 to 21 |
| | Batch Size (g) | 10 | 25 | 25 | 25 | 16 | 10 | 10 |
| Solvent | Dichloromethane (mL) | 50 | 250 | 250 | 50 | 32 | 100 | 50 |
| | Methanol (mL) | 50 | — | 250 | 200 | 128 | — | 50 |

TABLE 2

| | Experi- | Experi- | Experi- | Experi- | Experi- |
|---|---|---|---|---|---|
| | mental | mental | mental | mental | mental |
| Composition | group 1 | group 2 | group 3 | group 4 | group 5 |
| Lurasidone HCl | 16.7% | 14.3% | 33.3% | 8.3% | 10.0% |
| Polyvinyl acetate | 83.3% | 71.4% | 33.3% | 83.3% | 89.0% |
| phthalate (PVAP) | | | | | |
| Lauroyl Polyoxyl-32 | — | 14.3% | 33.3% | 8.3% | 1% |
| glycerides | | | | | |
| (product name: | | | | | |
| Gelucire 44/14) | | | | | |

TABLE 3

| | Experi- | Experi- | Experi- | Experi- |
|---|---|---|---|---|
| | mental | mental | mental | mental |
| Composition | group 6 | group 7 | group 8 | group 9 |
| Lurasidone HCl | 14.3% | 14.3% | 14.3% | 14.3% |
| PVAP | 71.4% | 71.4% | 71.4% | 71.4% |
| Admixture of | 14.3% | — | — | — |
| Lauroyl | | | | |
| Macrogol-32 | | | | |
| glycerides and | | | | |
| PEG 6000 | | | | |
| (product name: | | | | |
| Gelucire 59/14) | | | | |
| Stearoyl | — | 14.3% | — | — |
| polyoxyl-32 | | | | |
| glycerides | | | | |
| (product name: | | | | |
| Gelucire 50/13) | | | | |
| Linoleoyl | — | — | 14.3% | — |
| Polyoxyl-6 | | | | |
| glycerides | | | | |
| (product name: | | | | |
| Labrafil ® M2125 | | | | |
| CS) | | | | |
| Oleoyl polyoxyl-6 | — | — | — | 14.3% |
| glycerides | | | | |
| (Labrafil ® M1944 | | | | |
| CS) | | | | |

TABLE 4

| | Experi- | Experi- | Experi- | Experi- |
|---|---|---|---|---|
| | mental | mental | mental | mental |
| Composition | group 10 | group 11 | group 12 | group 13 |
| Lurasidone HCl | 14.3% | 14.3% | 14.3% | — |
| Lurasidone | — | — | — | 14.3% |
| PVAP | — | — | — | 71.4% |
| Hypromellose | 71.4% | — | — | — |
| acetate succinate | | | | |
| (HPMCAS) | | | | |
| Copovidone | — | 71.4% | — | — |
| Hydroxypropyl | — | — | 71.4% | — |
| Cellulose (HPC) | | | | |
| Lauroyl Polyoxyl- | 14.3% | 14.3% | 14.3% | 14.3% |
| 32 glycerides | | | | |
| (product name: | | | | |
| Gelucire 44/14) | | | | |

TABLE 5

| | Experi- | Experi- | Experi- | Experi- |
|---|---|---|---|---|
| | mental | mental | mental | mental |
| Composition | group 14 | group 15 | group 16 | group 17 |
| Lurasidone HCl | 14.3% | 12.5% | 12.5% | 12.5% |
| polyvinyl | 71.4% | — | 62.5% | 50.0% |
| caprolactam- | | | | |
| polyvinyl acetate- | | | | |
| polyethylene | | | | |
| glycol graft | | | | |
| copolymer | | | | |
| (Soluplus ®) | | | | |
| PVAP | — | 62.5% | 12.5% | 25.0% |
| hydroxypropyl | — | 12.5% | — | — |
| methylcellulose | | | | |
| (HPMC) | | | | |
| Lauroyl Polyoxyl- | 14.3% | 12.5% | 12.5% | 12.5% |
| 32 glycerides | | | | |
| (product name: | | | | |
| Gelucire 44/14) | | | | |

TABLE 6

| | Experi- | Experi- | Experi- | Experi- |
|---|---|---|---|---|
| | mental | mental | mental | mental |
| Composition | group 18 | group 19 | group 20 | group 21 |
| Lurasidone HCl | 14.3% | 14.3% | — | 14.3% |
| Lurasidone | — | — | 16.7% | — |
| Soluplus | 71.4% | — | — | — |
| PVAP | — | 71.4% | 83.3% | — |
| Cellulose acetate | — | — | — | 71.4% |
| phthalate (CAP) | | | | |
| Polyoxyethylated | 14.3% | — | — | — |
| 12-hydroxystearic | | | | |
| acid | | | | |
| (product name: | | | | |
| Kolliphor ® HS 15) | | | | |
| Glyceryl | — | 14.3% | — | — |
| monooleate | | | | |
| (product name: | | | | |
| Peceol ™) | | | | |
| Lauroyl Polyoxyl- | — | — | — | 14.3% |
| 32 glycerides | | | | |
| (product name: | | | | |
| Gelucire 44/14) | | | | |

TABLE 7
Spray drying parameter

| | |
|---|---|
| Inlet Temperature (° C.) | 70~90 |
| Outlet Temperature (° C.) | 35~60 |
| (Feed rate (mL/min) | 5~8 |

### Example 1.2—Preparation Method (Hot Melt Extrusion (HME))

The method of hot melt extrusion to prepare for solid dispersion including lurasidone is as follows:

Step 1, the active ingredient (lurasidone), the carrier and the surfactant are batch weighed according to table 8 to table 9, and then each group is blended evenly to obtain a mixture.

Step 2, the mixture obtained in step 1 is placed into a hot melt extruder (Pharma mini HME, Thermo) and then melted to obtain a hot melt extruded product. The hot melt extrusion parameters are shown in table 10.

Step 3, the hot melt extruded product is cooled to room temperature (for example, 25° C.) by air cooling for solidification. After solidification, the solidified product is crushed and then sieved with a 60-mesh sieve to obtain solid dispersion (experimental groups 22 to 28, respectively).

TABLE 8

| | Experimental | Experimental | Experimental |
|---|---|---|---|
| Composition | group 22 | group 23 | group 24 |
| Lurasidone | 14.3% | 33.3% | 14.3% |
| PVAP | 71.4% | 33.3% | 71.4% |
| Lauroyl Polyoxyl- | 14.3% | 33.3% | — |
| 32 glycerides | | | |
| (product name: | | | |
| Gelucire 44/14) | | | |
| Stearoyl polyoxyl- | — | — | 14.3% |
| 32 glycerides | | | |
| (product name: | | | |
| Gelucire 50/13) | | | |

TABLE 9

| | Experimental | Experimental | Experimental | Experimental |
|---|---|---|---|---|
| Composition | group 25 | group 26 | group 27 | group 28 |
| Lurasidone | 14.3% | 14.3% | 14.3% | 14.3% |
| HPMCAS | 71.4% | | — | — |
| Copovidone | — | 71.4% | — | |
| HPC | — | — | 71.4% | — |
| Polyvinyl | — | — | — | 71.4% |
| alcohol | | | | |
| (PVA) | | | | |
| Mesoporous | — | | — | 14.3% |
| silica | | | | |
| Lauroyl | 14.3% | 14.3% | 14.3% | — |
| Polyoxyl- | | | | |
| 32 glycerides | | | | |
| (product name: | | | | |
| Gelucire 44/14) | | | | |

TABLE 10
Hot melting extrusion parameter

| | |
|---|---|
| Screw speed (rpm) | 30~100 |
| Melting temperature (° C.) | 110~190 |

### Example 1.3—Dissolution and Storage Test

### 1.3.1—Dissolution Test 1

Dissolution tests of experimental group 1 to experimental group 28 produced by spray drying or hot melt extrusion, control group lurasidone HCl and lurasidone are performed to analyze the solubility of each experimental group and control group lurasidone HCl and lurasidone. The dissolution test is performed by paddle method in 500 mL of medium with a pH value of 6.0+0.01% SDS, in which a rotation speed is 100 rpm and a sampling time is 60 minutes. The results is shown in table 11.

TABLE 11

| | Solubility |
|---|---|
| Group | (μg/mL) |
| Lurasidone | 0.09 |
| Lurasidone | 0.46 |
| HCl | |
| Experimental | 1.3 |
| group 1 | |
| Experimental | 12.7 |
| group 2 | |
| Experimental | 2.5 |
| group 3 | |
| Experimental | 8.6 |
| group 4 | |
| Experimental | 5.1 |
| group 5 | |
| Experimental | 7.5 |
| group 6 | |
| Experimental | 2.8 |
| group 7 | |
| Experimental | 8.1 |
| group 8 | |
| Experimental | 11.0 |
| group 9 | |
| Experimental | 32.1 |
| group 10 | |
| Experimental | 33.7 |
| group 11 | |
| Experimental | 3.9 |
| group 12 | |
| Experimental | 8.7 |
| group 13 | |
| Experimental | 39.8 |
| group 14 | |
| Experimental | 19.3 |
| group 15 | |
| Experimental | 35.2 |
| group 16 | |
| Experimental | 52.4 |
| group 17 | |
| Experimental | 29.0 |
| group 18 | |
| Experimental | 26.2 |
| group 19 | |
| Experimental | 0.8 |
| group 20 | |
| Experimental | 2.3 |
| group 21 | |
| Experimental | 9.5 |
| group 22 | |
| Experimental | 1.0 |
| group 23 | |
| Experimental | 5.7 |
| group 24 | |
| Experimental | 2.4 |
| group 25 | |
| Experimental | 1.7 |
| group 26 | |
| Experimental | 2.0 |
| group 27 | |
| Experimental | 1.2 |
| group 28 | |

As shown in table 11, the 60-minute solubility of each experimental group is higher than control group (including only lurasidone or lurasidone HCl) in medium with a pH value of 6.0+0.01% SDS, and solubility of most experimental groups is higher than twice of solubility of control group. That is, solid dispersion of the experimental groups has improved solubility of lurasidone.

Furthermore, it is noticed that compared with experimental group 1 and experimental group 20 (lurasidone+PVAP, or lurasidone HCl+PVAP), experimental groups including surfactant (such as experimental groups 2-5, 13, 19) or experimental group including other specific carrier and surfactant (such as experimental groups 6-9) performs the higher solubility.

Moreover, as for experimental groups 22 and 23, it is also noticed that solubility increases (1.0 g/mL to 9.5 g/mL) when the concentration of the carrier (PVAP) increases (33.3% to 71.4%).

### Example 1.3.2—Dissolution Test 2 (Carrier Comparison: PVAP V.S. Crospovidone)

For comparing the applicability of the carriers suitable for solid dispersion including lurasidone, solid dispersion of comparative group 1 (including crospovidone, a kind of common carrier ingredient) is prepared according to Example 1.2 (HME) and the weight ratio of table 12, and the solubility is tested according the abovementioned method of Example 1.3.1. The comparative result is also listed in table 12.

TABLE 12

| | Weight Ratio | | | | |
|---|---|---|---|---|---|
| | | Lurasidone | Cros- | | Solubility |
| Prescription | Lurasidone | HCl | povidone | PVAP | (μg/mL) |
| Comparative | 1 | | 5 | | 0.42 |
| group 1 | | | | | |
| (prepared by HME) | | | | | |
| Experimental | 1 | | | ~5 | 0.8 |
| group 20 | | | | (4.99) | |
| Lurasidone | V | | | | 0.09 |
| Lurasidone HCl | | V | | | 0.46 |

Table 12 represents that solubility of comparative group 1 (the active ingredient is lurasidone; the carrier is crospovidone) is basically similar to lurasidone HCl. Relatively, solubility of experimental group 20 (the active ingredient is lurasidone; the carrier is PVAP) is significantly higher than lurasidone HCl and Comparative group 1 (the carrier is crospovidone). That is, the solid dispersion including PVAP served as carrier can achieve better solubility.

In addition, it is also noticed that in the preparation procedure of comparative group 1, the mixture including crospovidone is easily stuck in the hot melt extruder and hardly extruded, which causes difficulty in obtaining the solid dispersion.

Therefore, it should be emphasized that not all common carrier ingredients can be used in the solid dispersion including lurasidone, the carrier whether it is suitable for solid dispersion including lurasidone or lurasidone salts can only be confirmed after practical testing.

### Example 1.3.3—Storage Stability (Carrier: PVAP)

For observing storage stability of the experimental groups with different carriers or surfactants, experimental group 14 (no PAVP) and experimental group 16 (including PAVP) with similar solubility (experimental group 14: 39.8 μg/mL, experimental group 16: 35.2 μg/mL) are selected for the storage test. In detail, the experimental groups 14 and 16 are placed in 25° C. for 30 days and then analyzed by X-ray diffraction analysis (XRD) for observing the change of crystal structures.

The XRD results represent that after 30 days storage, experimental group 14 (no PAVP) has recrystallization issue, but experimental group 16 (including PAVP) after 30 days keeps amorphous state. Therefore, the solid dispersion including PAVP served as the carrier has better storage stability.

### Example 1.3.4—Dissolution Test 3 (Surfactant Comparison: Surfactant: Lauroyl Polyoxyl-32 Glycerides (Gelucire 44/14) V.S. Sodium Lauryl Sulfate (SLS))

For comparing the applicability of the surfactant suitable for solid dispersion including lurasidone, solid dispersions of comparative groups 2-1 to 2-4 (comparative groups 2-1 and 2-3 do not have surfactant; comparative groups 2-2 and 2-4 include SLS, a kind of common surfactant ingredient) are prepared according to Example 1.1 (spray drying) or Example 1.2 (HME) and the weight ratio of table 13 or table 14, and the solubility is tested according the abovementioned method of Example 1.3.1. The comparative result is also listed in table 13 and table 14.

TABLE 13
(spray drying)

| Solid Dispersion | | | | | |
|---|---|---|---|---|---|
| Prepared by Spray | Weight Ratio | | | | |
| drying Method of | Lurasidone | | | Gelucire | Solubility |
| Example 1.1 | HCl | HPMCAS | SLS | 44/14 | (μg/mL) |
| Comparative group | 1 | 5 | — | — | 1.3 |
| 2-1 | | | | | |
| Comparative group | 1 | 5 | 1 | — | 0.8 |
| 2-2 | | | | | |
| Experimental group 10 | 1 | ~5 | — | 1 | 32.1 |
| | | (4.99) | | | |

TABLE 14
(HME)

| Solid Dispersion | | | | | |
|---|---|---|---|---|---|
| Prepared by HME | Weight Ratio | | | | |
| Method of | | | | Gelucire | Solubility |
| Example 1.2 | Lurasidone | HPMCAS | SLS | 44/14 | (μg/mL) |
| Comparative group | 1 | 5 | — | — | 1.6 |
| 3-1 | | | | | |
| Comparative group | 1 | 5 | 1 | — | 2.0 |
| 3-2 | | | | | |
| Experimental group | 1 | ~5 | — | 1 | 2.8 |
| 25 | | (4.99) | | | |

Table 13 and table 14 represent that compared with solubility of comparative groups 2-1 to 2-4 (no surfactant, or the surfactant is SLS), solubility of experimental group 10 and 25 (the surfactant is Lauroyl Polyoxyl-32 glycerides (Gelucire 44/14)) is higher. That is, the solid dispersion including the Lauroyl Polyoxyl-32 glycerides served as the surfactant has better solubility.

Therefore, it should be noted that not all common surfactant ingredients can be used in the solid dispersion including lurasidone, the carrier suitable for solid dispersion including lurasidone or lurasidone salts can only be confirmed after practical testing.

### Example 1.4—Animal Test

Lurasidone HCl is used as control group, experimental groups 14-17, 22 and 28 are used as tested group, the pharmacokinetics of each group is evaluated in male Sprague-Dawley rats.

Specifically, control group and experimental groups 14-17, 22 and 28 are administrated to rats under fasting state, respectively, in which the dosage contains 10 mg/kg of lurasidone HCl or the amount of lurasidone equal to 10 mg/kg lurasidone HCl. The number of experimental rats in each group is three, and the sampling time is 0, 0.25, 0.5, 1, 1.5, 2, 4, 6, 8 and 24 hours for analyzing pharmacokinetic results, which are shown in table 15.

TABLE 15

| Experimental | | |
|---|---|---|
| group | Cmax (ng/mL) | AUC _{0-∞} (ng · hr/mL) |
| Lurasidone HCl | 32 | 102 |
| Experimental | 142 | 284 |
| group 14 | | |
| Experimental | 138 | 385 |
| group 15 | | |
| Experimental | 177 | 372 |
| group 16 | | |
| Experimental | 241 | 443 |
| group 17 | | |
| Experimental | 53 | 158 |
| group 22 | | |
| Experimental | 286 | 440 |
| group 28 | | |

Table 15 represents that maximum plasma concentration (Cmax) of lurasidone and area under curve (AUC) of each experimental group are higher than lurasidone HCl without being solid dispersed, which indicates the solid dispersions of the experimental groups including selected carrier and surfactant can achieve higher absorption efficiency. Therefore, food effect of lurasidone can be reduced or eliminated.

Additionally, it should be noted that experimental group 28 (carrier: PVA+mesoporous silica) performs a relatively high AUC (the highest Cmax and the second highest AUC) even though the solubility of which is only 1.2 μg/mL in Table 11 (the third lowest solubility in 28 experimental group groups). Therefore, the pairing that PVA and mesoporous silica can achieve unexpected and better absorption efficiency in animal study.

### Example 2—Pharmaceutical Composition

### 2. 1—Preparation Method

Experimental groups 2-1 to 2-3 (prepared by Experimental groups 2, 13 and 14, respectively)

Solid dispersions of the abovementioned experimental groups are manufactured into pharmaceutical compositions. The formulation is shown in table 16, which is solid and oral dosage form. The preparation method is as follows:

### 1. Manufacture of Tablet Core

(1) the materials except for magnesium stearate are blended to form intermixture according to the formulation in table 16 (table core).

(2) the intermixture in step (1) is sieved by using a 30 mesh sieve.

(3) the intermixture in step (2) is blended again.

(4) the intermixture in step (3) is blended with magnesium stearate to form core granules.

(5) the core granules in step (4) are pressed by using tablet machine to form a tablet core.

### 2. Manufacture of Outer Membrane

(1) materials of outer membrane in table 16 are dissolved in water to form a membrane solution with 15% (w/w) concentration.

(2) the membrane solution is sprayed on the tablet core obtained by the above step.

(3) the membrane is gained weight based on the formulation listed in table 16 to form film-coated tablets. The formulation proportion of outer membrane listed in table 16 are relative to the total weight of the tablet.

(4) film-coated tablets obtained in step (3) are dried in a film-coating machine at 45° C. for 15 minutes.

TABLE 16
(Experimental groups 2-1 to 2-3)

| Composition | | Experimental | Experimental | Experimental |
|---|---|---|---|---|
| (408 mg/Tablet) | | group 2-1 | group 2-2 | group 2-3 |
| Tablet | Lurasidone | Experimental | Experimental | Experimental |
| core | solid | group 2 | group 13 | group 14 |
| (w/w, %) | dispersion | 68.63 | 68.63 | 68.63 |
| | Mannitol | 25.74 | 25.74 | 25.74 |
| | Croscarmellose | 1.96 | 1.96 | 1.96 |
| | sodium | | | |
| | HPMC | 1.23 | 1.23 | 1.23 |
| | Magnesium | 0.49 | 0.49 | 0.49 |
| | Stearate | | | |
| Outer | Opadry pink | 1.96 | 1.96 | 1.96 |
| membrane | | | | |
| (w/w, %) | | | | |

### 2. 2—Dissolution Test

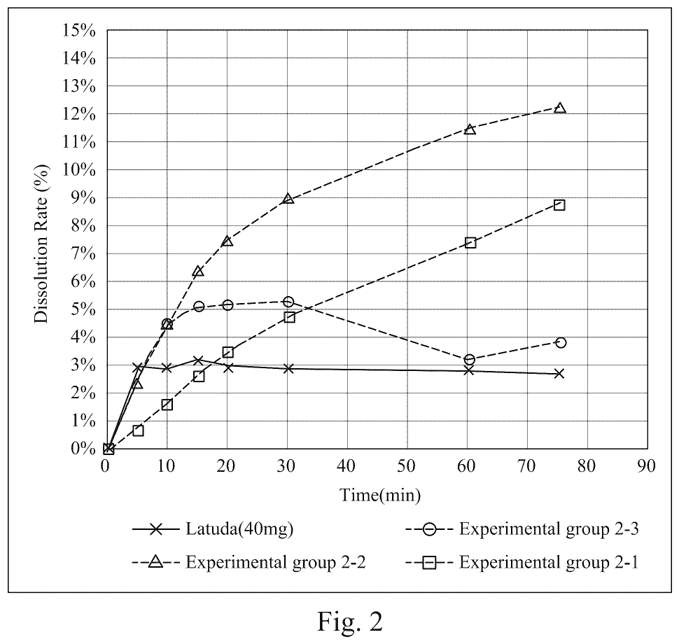

Dissolution tests of experimental group 2-1 to experimental group 2-3 are performed to analyze the solubility of each pharmaceutical composition of experimental groups and commercial product (product name: Latuda®). The dissolution test is performed by paddle method in 900 ml of medium with a pH value of 6.0+0.01% SDS, in which a rotation speed is 100 rpm for the first 60 minutes and 150 rpm for 60^{th} minute to 75^{th} minute, and a sampling time is 0, 5, 10, 15, 20, 30, 60 and 75 minutes, respectively. The results are showed in Table 17 and FIG. 2.

TABLE 17

| Time (min) | | 0 | 5 | 10 | 15 | 20 | 30 | 60 | 75 |
|---|---|---|---|---|---|---|---|---|---|
| Dissolution | Commercial | 0.0% | 2.9% | 2.9% | 3.2% | 2.9% | 2.8% | 2.8% | 2.7% |
| Rate | product | | | | | | | | |
| | Experimental | 0.0% | 0.7% | 1.6% | 2.6% | 3.4% | 4.7% | 7.4% | 8.8% |
| | group 2-1 | | | | | | | | |
| | Experimental | 0.0% | 2.3% | 4.4% | 6.4% | 7.4% | 8.9% | 11.4% | 12.2% |
| | group 2-2 | | | | | | | | |
| | Experimental | 0.0% | 2.7% | 4.5% | 5.1% | 5.2% | 5.3% | 3.2% | 3.8% |
| | group 2-3 | | | | | | | | |

Table 17 and FIG. 2 represent that compared with commercial product, experimental groups 2-2 and 2-3 perform the higher dissolution rate after 10 minutes and until 75 minutes, and experimental group 2-1 performs the higher dissolution rate after 20 minutes and until 75 minutes. It is noted that the pharmaceutical compositions prepared by the specific carrier and the specific surfactant in the present disclosure perform dissolution efficiency similar or even better than commercial product.

Although the disclosure has been disclosed in the above embodiments, it is not intended to limit the disclosure, and it is to be understood that those skilled in the art can make various changes and modifications without departing from the spirit and scope of the disclosure. The scope of protection of the present disclosure is subject to the definition of the scope of claims.

What is claimed is:

1. A solid dispersion, comprising:
lurasidone or its pharmaceutically acceptable salt; and
a carrier, wherein a material of the carrier comprises polyvinyl acetate phthalate, polyvinyl alcohol, cellulose acetate phthalate, mesoporous silica, hydroxypropyl methycellulose, polyvinyl caprolactam-polyvinyl acetate-polyethylene glycol graft copolymer, acrylic resin, hydroxypropyl cellulose, povidone, copovidone, ethyl cellulose, polyoxyethylene glycol, hypromellose acetate succinate, hypromellose phthalate, or a combination thereof.

2. The solid dispersion of claim 1, wherein a weight ratio of the lurasidone or its pharmaceutically acceptable salt to the carrier is from 3:1 to 1:30.

3. The solid dispersion of claim 1, wherein a weight percentage of the lurasidone or its pharmaceutically acceptable salt is from 3% to 75%, and a weight percentage of the carrier is from 25% to 97% based on 100% by weight percentage of the solid dispersion.

4. The solid dispersion of claim 1, wherein the carrier comprises a first carrier and a second carrier,
the first carrier comprises the polyvinyl acetate phthalate, the polyvinyl alcohol, the cellulose acetate phthalate, the mesoporous silica, the polyvinyl caprolactam-polyvinyl acetate-polyethylene glycol graft copolymer, the hydroxypropyl cellulose, the copovidone, the hypromellose acetate succinate, or a combination thereof, and
the second carrier comprises the polyvinyl alcohol, the mesoporous silica, the hydroxypropyl methycellulose, the polyvinyl caprolactam-polyvinyl acetate-polyethylene glycol graft copolymer, the acrylic resin, the hydroxypropyl cellulose, the povidone, the copovidone, the ethyl cellulose, the polyoxyethylene glycol, the hypromellose acetate succinate, the hypromellose phthalate, or a combination thereof.

5. The solid dispersion of claim 4, wherein a weight ratio of the lurasidone or its pharmaceutically acceptable salt to the first carrier and the second carrier is from 3:1 to 1:30.

6. The solid dispersion of claim 4, wherein a weight ratio of the first carrier to the second carrier is from 1:10 to 10:1.

7. The solid dispersion of claim 4, wherein a weight percentage of the lurasidone or its pharmaceutically acceptable salt is from 3% to 75%, a weight percentage of the first carrier and the second carrier is from 25% to 97% based on 100% by weight percentage of the solid dispersion.

8. The solid dispersion of claim 1, further comprising a surfactant, wherein the surfactant comprises glycerol esters, polyoxyethylene esters, or a combination thereof.

9. A pharmaceutical composition, comprising:
the solid dispersion of claim 1; and
an excipient.

10. A preparation method of a solid dispersion, comprising:
mixing lurasidone or its pharmaceutically acceptable salt and a carrier to form a mixture, wherein the carrier comprises polyvinyl acetate phthalate, polyvinyl alcohol, cellulose acetate phthalate, mesoporous silica, hydroxypropyl methycellulose, polyvinyl caprolactam-polyvinyl acetate-polyethylene glycol graft copolymer, acrylic resin, hydroxypropyl cellulose, povidone, copovidone, ethyl cellulose, polyoxyethylene glycol, hypromellose acetate succinate, hypromellose phthalate, or a combination thereof; and
spray drying the mixture, or
melting and then cooling the mixture.

11. The preparation method of claim 10, wherein the step of mixing comprises mixing the lurasidone or its pharmaceutically acceptable salt and the carrier in an organic solvent to form the mixture.

12. The preparation method of claim 10, wherein the step of melting comprises melting the mixture at a temperature of from 80° C. to 200° C. by using a hot melting extruder.

13. A solid dispersion, comprising:
lurasidone or its pharmaceutically acceptable salt;
a carrier; and
a surfactant, wherein the surfactant comprises glycerol esters, polyoxyethylene esters, or a combination thereof.

14. The solid dispersion of claim 13, wherein a weight ratio of the lurasidone or its pharmaceutically acceptable salt to the carrier is from 3:1 to 1:30.

15. The solid dispersion of claim 13, wherein a weight ratio of the lurasidone or its pharmaceutically acceptable salt to the surfactant is from 1:0.1 to 1:30.

16. The solid dispersion of claim 13, wherein a weight percentage of the lurasidone or its pharmaceutically acceptable salt is from 3% to 75%, a weight ratio of the carrier is from 25% to 97%, and a weight ratio of the surfactant is from 0.1% to 45% based on 100% by weight percentage of the solid dispersion.

17. The solid dispersion of claim 13, wherein the surfactant is fatty acid glycerides, polyoxyethylene fatty acid esters, or a combination thereof.

18. The solid dispersion of claim 17, wherein the surfactant polyoxylglycerides.

19. The solid dispersion of claim 13, wherein a material of the carrier comprises polyvinyl acetate phthalate, polyvinyl alcohol, cellulose acetate phthalate (CAP), mesoporous silica, hydroxypropyl methycellulose, polyvinyl caprolactam-polyvinyl acetate-polyethylene glycol graft copolymer, acrylic resin, hydroxypropyl cellulose, povidone, copovidone, ethyl cellulose, polyoxyethylene glycol, hypromellose acetate succinate, hypromellose phthalate, or a combination thereof.

20. The solid dispersion of claim 19, wherein the carrier comprises a first carrier and a second carrier,
the first carrier comprises the polyvinyl acetate phthalate, the polyvinyl alcohol, the cellulose acetate phthalate, the mesoporous silica, the polyvinyl caprolactam-polyvinyl acetate-polyethylene glycol graft copolymer, the hydroxypropyl cellulose, the copovidone, the hypromellose acetate succinate, or a combination thereof, and
the second carrier comprises the polyvinyl alcohol, the mesoporous silica, the hydroxypropyl methycellulose, the polyvinyl caprolactam-polyvinyl acetate-polyethylene glycol graft copolymer, the acrylic resin, the hydroxypropyl cellulose, the povidone, the copovidone, the ethyl cellulose, the polyoxyethylene glycol, the hypromellose acetate succinate, the hypromellose phthalate, or a combination thereof.
